(19) United States
(12) Patent Application Publication
(10) Pub. No.: US 2015/0209360 A1
(43) Pub. Date: Jul. 30, 2015

(54) ORAL CAFFEINE DELIVERY COMPOSITION

(71) Applicant: ORBZ, LLC, Lehi, UT (US)

(72) Inventors: Robert M. Reid, Lehi, UT (US); David Parish, Orem, UT (US)

(21) Appl. No.: 14/610,932

(22) Filed: Jan. 30, 2015

Related U.S. Application Data
(60) Provisional application No. 61/933,541, filed on Jan. 30, 2014.

(51) Int. Cl. A61K 31/522; A61K 9/16; A61K 9/48

(52) U.S. Cl. CPC A61K 31/522; A61K 9/4808; A61K 9/4825; A61K 9/4816; A61K 9/4875; A61K 9/4858; A61K 9/16; A61K 9/4833

(57) ABSTRACT

Apparatuses, systems, and methods are disclosed for an oral caffeine delivery composition. A liquid mixture comprises granulated caffeine crystals. A liquid mixture comprises an early-onset flavoring agent. A liquid mixture comprises a late-duration flavoring agent. A quick-dissolve shell comprises one or more layers encapsulating a liquid mixture.

### CROSS-REFERENCES TO RELATED APPLICATIONS

This application claims the benefit of U.S. Provisional Patent Application No. 61/933,541 entitled “ORAL CAFFEINE DELIVERY COMPOSITION” and filed on Jan. 30, 2014 for Robert M. Reid, et al., which is incorporated herein by reference.

### FIELD OF THE INVENTION

This invention relates to caffeine compositions and more particularly relates to masking bitterness of caffeine in an oral caffeine delivery composition.

### BACKGROUND

Caffeine is a bitter tasting crystalline xanthine alkaloid. Caffeine is a stimulant for the metabolic system and the central nervous system. Caffeine has been conventionally used both recreationally and medically to reduce physical fatigue and improve awareness and alertness. Although the effect of caffeine is different for each user, caffeine may also contribute to keeping users awake despite sleep deprivation conditions.

However, caffeine also has a bitter taste, making oral delivery of certain amounts of caffeine unpleasant without large amounts of other materials (e.g., soda, coffee) to dilute the bitter taste. Conventional caffeine consumption compositions, such as soda, coffee, energy drinks, and the like, although diluting the bitter taste, often take extra time for the caffeine to enter the bloodstream through digestion, as most or all of the caffeine is simply swallowed with the accompanying drink.

### SUMMARY

Oral caffeine delivery compositions are presented. In one embodiment, a liquid mixture comprises granulated caffeine crystals. In a further embodiment, a liquid mixture comprises an early-onset flavoring agent. A liquid mixture, in certain embodiments, comprises a late-duration flavoring agent. In an additional embodiment, a quick-dissolve shell comprises one or more layers which encapsulate a liquid mixture.

In one embodiment, a quick-dissolve shell is seamless. A quick-dissolve shell, in another embodiment, orally dissolves in less than about 30 seconds. A quick-dissolve shell, in a further embodiment, is substantially spherical and has a diameter of less than about 10 millimeters. In one embodiment, a quick-dissolve shell comprises a plurality of layers. For example, a quick-dissolve shell may comprise at least three layers. A quick-dissolve shell, in various embodiments, comprises a gelatin, lecithin, glycerin, and/or a sweetener.

In one embodiment, granulated caffeine crystals comprise less than 20% by mass of a composition. Granulated caffeine crystals, in a further embodiment, comprise between 3% and 9% by mass of a composition. In certain embodiments, granulated caffeine crystals each have a particle size less than about 300 micrometers.

In one embodiment, a late-duration flavoring agent comprises one or more of sucralose and xylitol. A late-duration flavoring agent, in a further embodiment, comprises less than 3% by mass of a composition. An early-onset flavoring agent, in certain embodiments, comprises an essential oil. In one embodiment, an essential oil comprises at least a peppermint essential oil.

A method of manufacturing an oral caffeine delivery composition is presented. A method, in one embodiment, includes preparing a liquid mixture. A liquid mixture, in certain embodiments, comprises granulated caffeine crystals. In a further embodiment, a liquid mixture comprises an early-onset flavoring agent. A liquid mixture, in one embodiment, includes a late-duration flavoring agent. A method, in a further embodiment, includes encapsulating a mixture in a quick-dissolve shell comprising one or more layers.

A mixture, in one embodiment, is seamlessly encapsulated in a quick-dissolve shell. Encapsulating a mixture in a quick-dissolve shell, in certain embodiments, comprises concentric dropping a mixture and a quick-dissolve shell using concentric nozzles coupled to concentric feed lines so that the quick-dissolve shell seamlessly encapsulates the mixture.

Another oral caffeine delivery composition is presented. A liquid mixture, in one embodiment, comprises granulated caffeine crystals each having a particle size less than about 300 micrometers. A liquid mixture, in a further embodiment, comprises an early-onset flavoring agent comprising an essential oil. In one embodiment, a liquid mixture comprises a late-duration flavoring agent comprising less than 3% by mass of a composition. A dissolvable shell, in certain embodiments, includes at least three layers so that an essential oil does not dissolve through a shell for a shelf life of at least a year.

In one embodiment, a dissolvable shell comprises a gelatin, lecithin, glycerin, and/or a portion of a late-duration flavoring agent comprising a sweetener. Granulated caffeine crystals, in certain embodiments, comprise between 3% and 9% by mass of the composition. An essential oil, in a further embodiment, comprises between 20% and 40% by mass of the composition.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Discussion of the features and advantages, and similar language, throughout this specification may, but do not necessarily, refer to the same embodiment.

The described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description, or may be learned by the practice of the invention as set forth hereinafter.

### BRIEF DESCRIPTION OF THE DRAWINGS

In order that the advantages of the subject matter of the present disclosure will be readily understood, a more particular description of the subject matter will be rendered by reference to specific embodiments that are illustrated in the appended drawings. Understanding that these drawings depict only typical embodiments of the subject matter of the present disclosure and are not therefore to be considered to be limiting of its scope, the subject matter will be described and explained with additional specificity and detail through the use of the accompanying drawings, in which:

FIG. 1A is a cross-sectional view of one embodiment of a mixture encapsulated within a quick-dissolve shell;

FIG. 1B is another cross-sectional view of one embodiment of a mixture encapsulated within a multi-layer quick-dissolve shell;

FIG. 2A is a schematic block diagram of a composition of the mixture depicted in FIGS. 1A and 1B, according to one embodiment;

FIG. 2B is a schematic block diagram of a composition of the mixture depicted in FIGS. 1A and 1B, according to another embodiment;

FIG. 3A depicts a user orally taking a caffeine delivery composition, according to one embodiment; and

FIG. 3B depicts the oral caffeine delivery composition once the shell has dissolved in the mouth of a user.

### DETAILED DESCRIPTION

The subject matter of the present disclosure has been developed in response to the present state of the art in caffeine delivery systems. Accordingly, the subject matter of the present disclosure has been developed to provide a composition for orally delivering caffeine that overcomes many or all or some shortcomings in the prior art.

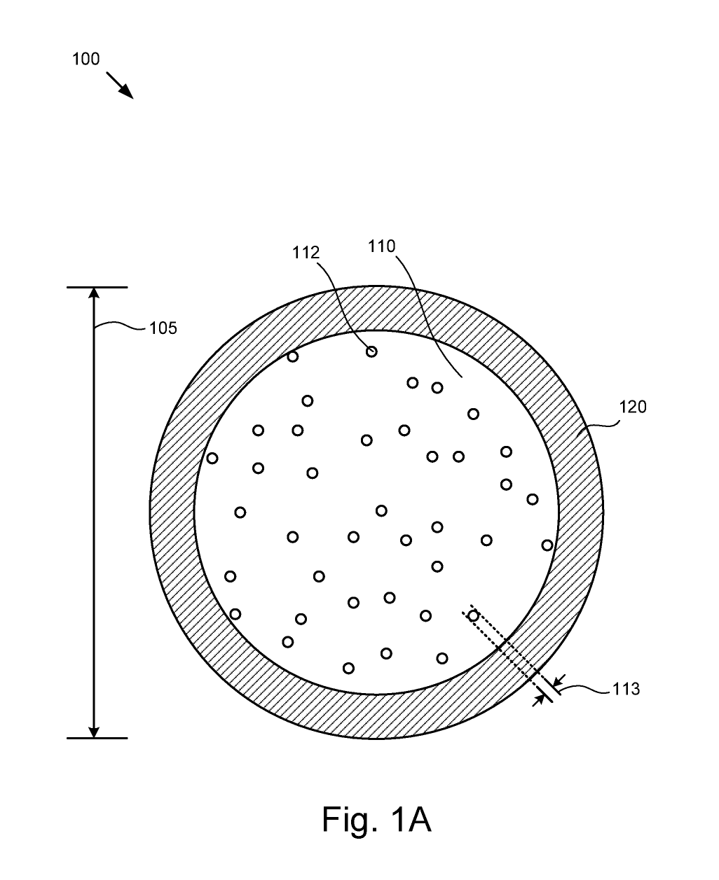

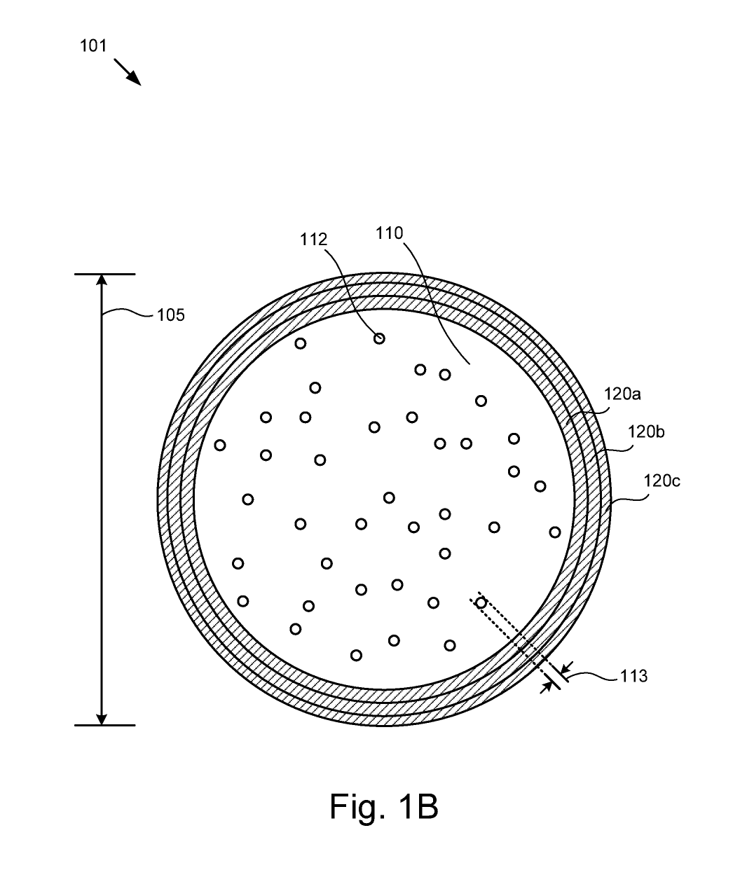

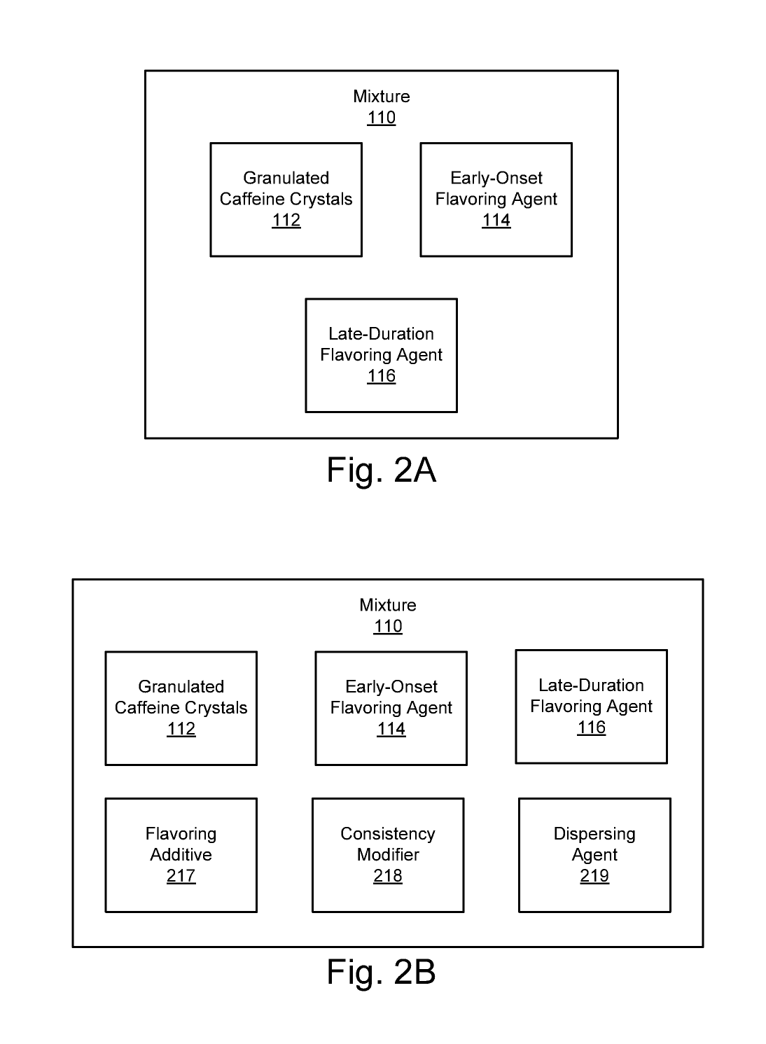

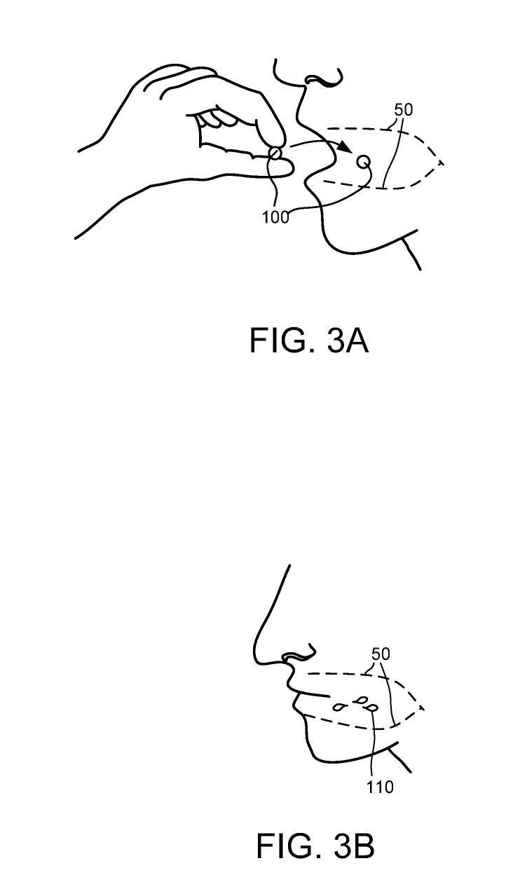

FIG. 1A is a cross-sectional view of one embodiment of an oral caffeine delivery composition 100 that includes a mixture 110 encapsulated within a quick-dissolve shell 120. Generally, the oral caffeine delivery composition 100 of the present disclosure is configured to be inserted within the mouth of a user to dissolve and disintegrate the quick-dissolve shell 120, thereby exposing the mouth and tongue to the mixture 110. The mixture 110 includes, among other constituents, granulated caffeine crystals 112 that are rapidly introduced into the bloodstream via the mouth of the user. However, since caffeine has a bitter taste, the mixture 110 further includes one or more flavoring agents that mask the taste of the caffeine. Additional details regarding the mixture 110 are included below with reference to FIGS. 2A and 2B and additional details regarding a method of using the oral caffeine delivery composition 100 are included below with reference to FIG. 3A and 3B.

The quick-dissolve shell 120 may be made from a variety of materials and may be manufactured according to various procedures. Generally, the quick-dissolve shell 120 is configured to rapidly dissolve in the mouth of a user, thus allowing the encapsulated mixture 110 to disperse throughout the mouth and across the tongue in order for the granulated caffeine crystals 112 to enter the bloodstream of the user. The granulated caffeine crystals 112 are depicted in Figure as circular particles dispersed throughout the mixture 110. However, the granulated caffeine crystals 112 may have other shapes, sizes 113, and relative positions. In other words, the shape, relative size 113, and spatial density of the granulated caffeine crystals 112 depicted in FIGS. 1A and 1B are not intended to limit the scope of the present disclosure. Additional details regarding the granulated caffeine crystals are included below with reference to FIG. 2A.

The quick-dissolve shell 120, according to one embodiment, may be made from a gelatinous material that is specifically selected according to its ability to rapidly dissolve under certain aqueous temperature conditions (i.e., the temperature of the saliva within a user's mouth). For example, the quick-dissolve shell 120 may be made from gelatin that has a bovine, porcine, fish, and/or poultry origin (e.g., porcine gelatin, fish gelatin, a combination of porcine and fish gelatin, or the like). In another embodiment, the quick-dissolve shell 120 may be constructed from agar or other plant-based gelatinous substances. In such embodiments, an additional additive may be implemented into the shell 120 in order to facilitate the rapid dissolution of the shell 120 in the mouth of a user. For example, agar does not readily dissolve at temperatures near the human body temperature, thus a pH modifying agent may also be incorporated into the shell 120 to facilitate the dissolving reaction.

According to one embodiment, the shell 120 may further include a plasticizer agent, such as starch or amylum acetate. The plasticizer may be chosen from the group comprising polyols, in particular glycerol (e.g., glycerin), xylitol, sorbitol, polyglycerol, non-crystallisable solutions of sorbitol, glucose, fructose and glucose syrups, among others. One embodiment of a plasticizer is a mixture of sorbitol, sorbitans, maltitol, and mannitol sold under the brand name ANIDRISORB® by Roquette. Depending on the specifics of a given application (e.g., depending on the volume/dose of the mixture 110 contained within the shell 120), the composition of the shell 120 may be selected to produce an oral caffeine delivery mechanism that delivers the caffeine into the bloodstream at the proper rate. In one embodiment, the shell may be specifically configured to be harder so that a user must chew on the shell 120 (at least initially) to begin dissolving the constituents.

The shell 120, in certain embodiments, may include a flavoring agent (e.g., at least a portion of an early-onset flavoring agent, at least a portion of a late-duration flavoring agent, a sweetener, or the like). For example, in one embodiment, the late-duration flavoring agent comprises sucralose or the like, and the shell 120 may comprise at least a portion of the late-duration flavoring agent, along with the mixture 110 within the shell 120. In this manner, since the late-duration flavoring agent may take longer for the user to taste or detect, including a portion of the late-duration flavoring agent in the shell 120 may decrease an amount of time until the user may taste the late-duration flavoring agent, as the user may begin tasting the late-duration flavoring agent before the shell 120 has dissolved and the mixture 110 has ruptured the shell 120. The shell 120 and/or the mixture 110, in a further embodiment, may include a coloring agent to provide an aesthetically pleasing color to the composition 100.

FIG. 1B depicts one embodiment of an oral caffeine delivery composition 101 that includes a mixture 110 encapsulated within a quick-dissolve shell 120 comprising multiple layers 120a-c. In certain embodiments, one or more layers 120a-c of the shell 120 may include a coating or an additional layer 120a-c specifically designed to prevent the contained mixture 110 from pre-mature leaking. For example, each layer 120a-c may deposited in separate step or process, such as the ‘concentric dropping’ manufacturing process described below, depositing different layers in different steps.

The number and/or thickness of layers 120a-c may be determined or selected based on chemical or other physical properties of the mixture 110 (e.g., an acidity, a chemical reactivity, or the like) to ensure that the mixture 110 does not dissolve through or rupture the layers 120a-c of the shell 120 for at least a predefined shelf life, such as a shelf life of at least a year, at least two years, or another predefined threshold. In one embodiment, the shell 120 comprises two layers 120a-b, at least three layers 120a-c, or more layers 120a-c, such that a mixture 110 comprising an essential oil (e.g., a peppermint essential oil) does not dissolve through or rupture the layers 120a-c for a shelf life of at least a year.

FIGS. 1A and 1B depict the quick-dissolve shell 120 as having a spherical shape. In other embodiments, the quick-dissolve shell 120 may have a cylindrical or pill shape. In yet other embodiments, the quick-dissolve shell 120 may resemble a lozenge or may have an ovular shape. Additionally, the thickness of the walls of the shell 120 and the overall size 105 of the oral caffeine delivery composition 100 may vary according to the specifics of a given application. For example, in one embodiment the material of the shell 120 only constitutes less than about 15% of the total mass of the oral caffeine delivery composition 100. In yet another embodiment, the mass percentage of the material of the shell 120 is between about 5% and 30% of the total mass of the oral caffeine delivery composition 100.

In one embodiment, the shell 120 may be manufactured in such a manner that the shell 120 is substantially seamless. In other words, the quick-dissolve shell 120 and the mixture 110 may be substantially simultaneously formed, thus eliminating the need to couple two halves of a capsule together at a seam to form the shell 120. In such an embodiment, a ‘concentric dropping’ manufacturing method may be implemented. In other words, concentric nozzles directed in a substantially downward direction may be coupled to concentric feed lines. As liquid drips from the concentric nozzles, a first liquid dripping from the annular nozzle encapsulates a second liquid dripping from the center nozzle, thus forming a seamless droplet of liquid with an outer-shell made from the first liquid and an interior mixture made of the second liquid.

The diameter 105 of the quick-dissolve shell 120, according to one embodiment, may be less than 20 millimeters. In another embodiment, the diameter 105 of the quick-dissolve shell 120 is between about 2 millimeters and 10 millimeters. In yet another embodiment, the diameter 105 of the quick-dissolve shell 120 is about 6 millimeters (e.g., between about 5.5 millimeters and 6.5 millimeters, or the like). The oral caffeine delivery composition 100 may be sized according to the expected or anticipated user. For example, the volume of the mixture 110 contained within the quick-dissolve shell 120 and the thickness of the shell 120 wall may depend on the age, weight, and/or preferences of a user. For example, if a user would like a comparatively larger dose of caffeine, a comparatively larger shell 120 could be implemented to hold the larger volume of mixture 110.

The dissolving rate of the shell 120 is dependent on a variety of factors, including the diameter 105 of the composition 100, the thickness of the shell wall 120 and/or number of layers 120a-c, and/or the material used to construct the shell 120. In one embodiment, the quick-dissolve shell 120 dissolves, at least partially (e.g., ruptures) in less than about 1 minute. In another embodiment, the quick-dissolve shell 120 dissolves in less than about 30 seconds. In yet another embodiment, the quick-dissolve shell 120 dissolves in about 10 seconds, or less.

Not only does the shell 120 directly affect the volume of the mixture 110 and the delivery rate of the mixture 110 to the mouth of a user, the quick-dissolve shell 120 may provide other benefits as well. For example, the shell 120 may be acid resistant or resistant to freezing. In other words, it is possible to greatly improve the storage time of substances that would be oxidized if exposed to air or substances whose qualities change when exposed to light or moisture. Additionally, the shell 120 also allows low-boiling point substances (e.g., flavors, additives—discussed below) to be stored for long periods of time without evaporating or oxidizing.

FIG. 2A is a schematic block diagram of a composition of the mixture 110 depicted in FIGS. 1A and 1B, according to one embodiment. The mixture 110 includes granulated caffeine crystals 112, an early-onset flavoring agent 114, and a late-duration flavoring agent 116. While these constituents are described in detail below, the granulated caffeine crystals 112 may generally be in solid form and may be suspended/mixed with the early-onset flavoring agent 114 and the late-duration flavoring agent 116. As briefly described above, once the composition 100 has been placed in the mouth of a user and the quick-dissolve shell 120 has dissolved/disintegrated, the mixture 110 disperses through the mouth of a user and/or is swallowed, and the granulated caffeine crystals 112 are absorbed into the bloodstream of a user.

Caffeine is a bitter tasting crystalline xanthine alkaloid. Caffeine is a stimulant for the metabolic system and the central nervous system. Caffeine has been conventionally used both recreationally and medically to reduce physical fatigue and improve awareness and alertness. Although the effect of caffeine is different for each user, caffeine may also contribute to keeping users awake despite sleep deprivation conditions. Caffeine may also have other effects on the body, such as increased muscle performance and improved brain function. Further, caffeine has been associated with a lower overall risk of cancer and other diseases. However, caffeine may also have some negative side-effects, depending on the specific health status of a certain user and/or if consumed in large amounts.

Therefore, the present disclosure implements an oral caffeine delivery composition 100 that delivers a specified amount of caffeine in an efficient and controllable manner. Since the granulated caffeine crystals 112 are rapidly introduced into the bloodstream via the mouth, the beneficial effects of caffeine may be somewhat immediately experienced by the user. In other words, the oral caffeine delivery composition 110 of the present disclosure provides caffeine in a substantially elemental form and thus allows for caffeine to be substantially immediately administered. In contrast, conventional caffeine consumption compositions, such as coffee, energy drinks, etc., require extra time for the caffeine to enter the bloodstream through digestion.

The granulated caffeine crystals 112, according to one embodiment, are crystalline structures of caffeine that have been ground to a desired size. In one embodiment, the granulated caffeine crystals 112, as briefly described above with reference to FIG. 1A, are manufactured to be small enough so as to have a smooth-mouth feel when the mixture 110 is dispersed throughout the mouth of a user. In other words, the crystalline particle size of the individual granulated caffeine crystals 112 is smaller than about 300 micrometers. In another embodiment, the granulated caffeine crystals 112 are each between about 150 and 250 micrometers. In yet another embodiment, the granulated caffeine crystals 112 are each about 220 micrometers. Moreover, particle size distribution may have an effect on the strength of the shell 120. For example, encapsulation of larger particles can create shells 120 that are not substantially strong, or the like.

The granulated caffeine crystals 112 may be prepared according to a variety of manufacturing methods. In one embodiment, bulk crystalline caffeine is ground and sieved into granulated particles that have the proper size. The size of the individual granulated caffeine crystals 112 may also be important for promoting absorption of the caffeine into the bloodstream. While the amount of caffeine within the mixture 110 and within each shell 120 is dependent on the strength of the dose, in one embodiment the granulated caffeine crystals 112 comprise less than about 50% by mass of the composition 100. In another embodiment, the granulated caffeine crystals 112 comprise less than about 20% by mass of the composition 100. In a further embodiment, the granulated caffeine crystals 112 comprise less than about 10% by mass of the composition 100 (e.g., between about 3% and 9%, about 6%, or the like by mass of the composition). In one implementation, the composition 100 includes between about 4 mg and 8 mg of caffeine (e.g., about 6 mg), and other constituents 114, 116 and shell 120 properties are configured accordingly.

In certain embodiments, by including a relatively low dose of caffeine crystals 112, a user may easily consume a determined dosage level by selecting a number of compositions 100 to consume (e.g., one user may desire a lower dose and may consume one, another user may desire a higher dose and may consume several). In another embodiment, it may be desirable to include a higher dose of caffeine crystals 112 in the composition 100, to decrease the amount of compositions 100 required to achieve a desired dosage level, or the like.

The early-onset flavoring agent 114 is included in the mixture 110 to substantially immediately mask the bitter taste of caffeine. As soon as the quick-dissolve shell 120 is ruptured or dissolves away, the mixture 110 contained within the shell 120 is released and disperses through the mouth and across the tongue. The early-onset flavoring agent 114 may have strong flavor to substantially overload the tongue and the taste buds with the taste/flavor of the early-onset flavoring agent 114, thus preventing the tongue and taste-buds from receiving/perceiving the bitter taste of the granulated caffeine crystals 112. Depending on the specifics of a given application, the early-onset flavoring agent 114 may be in solid or liquid form and the mass percentage of the early-onset flavoring agent 114 may be between about 1% and about 90% of the mass of the composition 100.

Since essential oils are often powerful and fast-acting agents, the early-onset flavoring agent 114, in certain embodiments, may comprise an essential oil. Examples of essential oils which may comprise an early-onset flavoring agent 114 include peppermint essential oil (e.g., mentha piperita), wintergreen essential oil, spearmint essential oil, anise essential oil, cinnamon essential oil, clove or clove leaf essential oil, nutmeg essential oil, lemon essential oil, lime essential oil, orange essential oil, grapefruit essential oil, lemongrass essential oil, tangerine essential oil, one or more other essential oils, and/or mixtures thereof, among others.

Additionally or instead of one or more essential oils, in one embodiment, the early-onset flavoring agent 114 may include a spice, extract, concentrate, or other powerful flavor, which may or may not be a product of and/or derived from an essential oil, such as pulegone, maltol, menthol, fruit flavorings, aniseed, caramel, honey, liquorice, cream, various spices and combinations thereof with other flavorings, and mixtures thereof, among others. In one specific embodiment, the early-onset flavoring agent 114 is a peppermint essential oil product (e.g., mentha piperita).

In certain embodiments, the early-onset flavoring agent 114 (e.g., peppermint essential oil or the like) constitutes between about 0.1% and 50% by mass of the composition 100. In a further embodiment, the early-onset flavoring agent 114 (e.g., peppermint essential oil or the like) constitutes between about 20% and 40% by mass of the composition 100. In one embodiment, the early-onset flavoring agent 114 (e.g., peppermint essential oil or the like) constitutes between about 25% and 35% by mass of the composition 100 (e.g., about 30% by mass of the composition 100 or the like).

The late-duration flavoring agent 116 is included in the mixture 110 to mask the long-lasting bitter taste of caffeine. Caffeine not only has an immediately bitter flavor, but the bitter taste can remain in the mouth of a user for some time after initially contacting the tongue. Therefore, once the early-onset flavoring agent 114 has been absorbed into the bloodstream and/or its flavor has dissipated, a longer lasting flavor may be used to mask the ongoing bitter taste of caffeine. Thus, the late-duration flavoring agent 116 may be included in the mixture 110 to provide the long term (e.g., several minutes) taste masking of the caffeine.

The late-duration flavoring agent 116 may have a strong flavor that coats portions of the mouth and tongue to continually provide the masking flavor. For example, a strong sweetener may be used as the late-duration flavoring agent 116, in certain embodiments. Depending on the specifics of a given application, the late-duration flavoring agent 116 may be in solid or liquid form and the mass percentage of the late-duration flavoring agent 114 may be between about 1% and about 90% of the mass of the composition 100.

Since sweeteners are often powerful and long-lasting agents, the late-duration flavoring agent 116 may be a sweetener. Examples of non-sugar sweeteners which may be used as a late-duration flavoring agent 116 include one or more of sucralose, saccharin, sodium saccharinate, stevia, acesulpham K, neohesperidin hydrochloride, mannitol, xylitol, maltitol, sorbitol, sodium cyclamate, mogrosides, aspartame, menthol and mixtures thereof, among others. In another embodiment, the late-duration flavoring agent 116 may comprise a sugar (e.g., sucrose, glucose, fructose, or the like). Certain non-sugar sweeteners or sugar substitutes may have many times the sweetness of sugar. For example, sucralose may be about 600 times as sweet as sugar. Using sucralose or another non-sugar sweetener as the late-duration flavoring agent 116 may provide a high level of sweetness and flavor, even in a shell 120 having a small diameter.

The late-duration flavoring agent 116 (e.g., sucralose or another sweetener), in one embodiment, constitutes between 0.1% and 90% by mass of the composition 100. In a further embodiment, the late-duration flavoring agent 116 (e.g., sucralose or another sweetener), constitutes between 0.1% and 50% by mass of the composition 100. The late-duration flavoring agent 116 (e.g., sucralose or another sweetener), in another embodiment, constitutes between 0.1% and 20% of the composition 100. In one embodiment, the late-duration flavoring agent 116 (e.g., sucralose or another sweetener), constitutes less than about 10% by mass of the composition 100. In certain embodiments, the late-duration flavoring agent 116 (e.g., sucralose or another sweetener), constitutes less than about 5% by mass of the composition 100. The late-duration flavoring agent 116 (e.g., sucralose or another sweetener), in one embodiment, comprises less than about 1% by mass of the composition 100. As described above, in certain embodiments, at least a portion of the late-duration flavoring agent 116 may be in the shell 120, in addition to or instead of in the mixture 110.

FIG. 2B is a schematic block diagram of a composition of the mixture 110 depicted in FIGS. 1A and 1B, according to another embodiment. The mixture 110 depicted in FIG. 2B includes granulated caffeine crystals 112, an early-onset flavoring agent 114, a late-duration flavoring agent 116, a flavoring additive 217, a consistency modifier 218, and a dispersing agent 219. The granulated caffeine crystals 112, early-onset flavoring agent 114, and late-duration flavoring agent 116 are described above with reference to FIG. 2A. The additional constituents are described below.

The flavoring additive 217 may be an additional flavoring agent that provides the overall mixture 110 with a specific taste. For example, in certain implementations the oral caffeine delivery composition 100 may be marketed and packaged with different flavors, such as a licorice flavor, a cinnamon flavor, a chocolate favor, etc. Thus, the flavoring additive 217, while it may also contribute to masking the bitter taste of caffeine, may be included in the mixture 110 as the overall flavor of the composition 100.

The consistency modifier 218, according to one embodiment, may be a constituent that affects the liquidity, viscosity, and/or solubility of the mixture 110. In other words, depending on the specific size and concentration of the granulated caffeine crystals 112 and/or the composition and state of the selected early-onset flavoring agent 114 and the late-duration flavoring agent 116, the overall consistency of the mixture 110 may need to be altered or modified. For example, the consistency modifier 218 may include water to dilute the mixture 110, thus making dispersion of the flavoring agents 114, 116 and the caffeine crystals 112 more efficient. In another embodiment, the consistency modifier 218 may include a vegetable oil or other naturally occurring oil, such as coconut oil. The consistency modifier 218, in various embodiments, may comprise between about 1% and 90% of the composition 100, between about 25% and 75% of the composition 100, between about 40% and 60% of the composition, about 50% of the composition, or the like, by mass.

The mixture 110 may further include a dispersing agent 219 that functions to facilitate the suspension and/or mixture of the solid granulated caffeine crystals 112 in the mixture 110. For example, the granulated caffeine crystals 112 may be coated with a dispersing agent 219 to give the caffeine improved solubilization and disintegration properties. According to a particular embodiment, the dispersing agent 219 may include excipients selected from the group comprising effervescent agents, super disintegrants, lipophilic surfactants, hydrophilic surfactants, and hydrosoluble agents promoting dispersion, among others.

FIG. 3A depicts a user orally taking caffeine delivery composition 100, according to one embodiment. As described above and throughout the present disclosure, a user may place the composition 100 (the shell 120 and the mixture 110 contained within the shell 120) in his mouth and the shell 120 may begin to dissolve/disintegrate. The size of the composition 100 depicted in FIG. 3A is not intended as an accurate scale depiction of the size of the composition 100 with respect to the user's hand or the mouth 50 of the user. In certain embodiments, as described above, the composition 100 may be much smaller than depicted (e.g., on the scale of a few millimeters).

FIG. 3B depicts the oral caffeine delivery composition 100 after it has dissolved in the mouth 50 of a user. Once again, as described above, once the shell 120 has dissolved/disintegrated, the mixture 110 disperses throughout the mouth 50 of the user and the granulated caffeine crystals 112 are absorbed into the bloodstream. The bitter taste of the caffeine is masked, both immediately and over time by the earl-onset flavoring agent 114 and the late-duration flavoring agent 116, respectively.

Reference throughout this specification to features, advantages, or similar language does not imply that all of the features and advantages that may be realized with the subject matter of the present disclosure should be or are in any single embodiment of the subject matter. Rather, language referring to the features and advantages is understood to mean that a specific feature, advantage, or characteristic described in connection with an embodiment is included in at least one embodiment of the subject matter of the present disclosure. Thus, discussion of the features and advantages, and similar language, throughout this specification may, but do not necessarily, refer to the same embodiment.

Furthermore, the described features, structures, advantages, and/or characteristics of the subject matter of the present disclosure may be combined in any suitable manner in one or more embodiments and/or implementations. In the following description, numerous specific details are provided to impart a thorough understanding of embodiments of the subject matter of the present disclosure. One skilled in the relevant art will recognize that the subject matter of the present disclosure may be practiced without one or more of the specific features, details, components, materials, and/or methods of a particular embodiment or implementation. In other instances, additional features and advantages may be recognized in certain embodiments and/or implementations that may not be present in all embodiments or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims, or may be learned by the practice of the subject matter as set forth hereinafter.

Similarly, reference throughout this specification to “one embodiment,” “an embodiment,” or similar language means that a particular feature, structure, or characteristic described in connection with the embodiment is included in at least one embodiment of the subject matter of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the subject matter of the present disclosure, however, absent an express correlation to indicate otherwise, an implementation may be associated with one or more embodiments.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. An oral caffeine delivery composition comprising:
a liquid mixture comprising
granulated caffeine crystals,
an early-onset flavoring agent, and
a late-duration flavoring agent; and
a quick-dissolve shell comprising one or more layers, wherein the quick-dissolve shell encapsulates the mixture.

2. The oral caffeine delivery composition of claim 1, wherein the quick-dissolve shell is seamless.

3. The oral caffeine delivery composition of claim 1, wherein the quick-dissolve shell orally dissolves in less than about 30 seconds.

4. The oral caffeine delivery composition of claim 1, wherein the quick-dissolve shell is substantially spherical and has a diameter of less than about 10 millimeters.

5. The oral caffeine delivery composition of claim 1, wherein the quick-dissolve shell comprise a plurality of layers.

6. The oral caffeine delivery composition of claim 1, wherein the quick-dissolve shell comprises at least three layers.

7. The oral caffeine delivery composition of claim 1, wherein the quick-dissolve shell comprises a gelatin, lecithin, glycerin, and a sweetener.

8. The oral caffeine delivery composition of claim 1, wherein the granulated caffeine crystals comprise less than 20% by mass of the composition.

9. The oral caffeine delivery composition of claim 5, wherein the granulated caffeine crystals comprises between 3% and 9% by mass of the composition.

10. The oral caffeine delivery composition of claim 1, wherein the granulated caffeine crystals each have a particle size less than about 300 micrometers.

11. The oral caffeine delivery composition of claim 1, wherein the late-duration flavoring agent comprises one or more of sucralose and xylitol.

12. The oral caffeine delivery composition of claim 8, wherein the late-duration flavoring agent comprises less than 3% by mass of the composition.

13. The oral caffeine delivery composition of claim 1, wherein the early-onset flavoring agent comprises an essential oil.

14. The oral caffeine delivery composition of claim 10, wherein the essential oil comprises at least a peppermint essential oil.

15. A method of manufacturing an oral caffeine delivery composition, the method comprising:
preparing a liquid mixture comprising
granulated caffeine crystals,
an early-onset flavoring agent, and
a late-duration flavoring agent; and
encapsulating the mixture in a quick-dissolve shell comprising one or more layers.

16. The method of claim 15, wherein the mixture is seamlessly encapsulated in the quick-dissolve shell.

17. The method of claim 15, wherein encapsulating the mixture in the quick-dissolve shell comprises concentric dropping the mixture and the quick-dissolve shell using concentric nozzles coupled to concentric feed lines such that the quick-dissolve shell seamlessly encapsulates the mixture.

18. An oral caffeine delivery composition comprising:
a liquid mixture comprising
granulated caffeine crystals each having a particle size less than 300 micrometers,
an early-onset flavoring agent comprising an essential oil, and
a late-duration flavoring agent comprising less than 3% by mass of the composition; and
a dissolvable shell comprising at least three layers such that the essential oil does not dissolve through the shell for a shelf life of at least a year, wherein the quick-dissolve shell encapsulates the mixture.

19. The oral caffeine delivery composition of claim 18, wherein the dissolvable shell comprises a gelatin, lecithin, glycerin, and a portion of the late-duration flavoring agent, the late-duration flavoring agent comprising a sweetener.

20. The oral caffeine delivery composition of claim 18, wherein the granulated caffeine crystals comprises between 3% and 9% by mass of the composition and the essential oil comprises between 20% and 40% by mass of the composition.
